(19) United States
(12) Patent Application Publication
(10) Pub. No.: US 2025/0160846 A1
(43) Pub. Date: May 22, 2025

(54) MEDICAL SYSTEMS, DEVICES, AND METHODS FOR DELIVERING ONE OR MORE TREATMENT AGENTS

(71) Applicant: Boston Scientific Scimed, Inc., Maple Grove, MN (US)

(72) Inventor: Rosangel Aimee Ramos ESPINOZA, Allston, MA (US)

(73) Assignee: Boston Scientific Scimed, Inc., Maple Grove, MN (US)

(21) Appl. No.: 18/950,695

(22) Filed: Nov. 18, 2024

Related U.S. Application Data
(60) Provisional application No. 63/601,449, filed on Nov. 21, 2023.

(51) Int. Cl. A61B 17/12; A61B 17/00

(52) U.S. Cl. CPC A61B 17/12186; A61B 17/00491; A61B 2017/12004; A61B 2017/1205

(57) ABSTRACT

Disclosed is a medical device comprising a sleeve; a conveyor movable relative to the sleeve; a treatment agent disposed within a cavity defined by the conveyor; and an actuator coupled to a first portion of the conveyor, wherein the actuator is movable relative to the sleeve; wherein: a second portion of the conveyor is movable from a first position radially within the sleeve to a second position radially exterior to the sleeve, and at least a portion of the treatment agent is movable from a first position within the cavity to a second position outside of the cavity.

### CROSS-REFERENCE TO RELATED APPLICATIONS

This application claims the benefit of priority of U.S. Provisional Patent Application No. 63/601,449 filed Nov. 21, 2023, the entirety of which is incorporated herein by reference.

### TECHNICAL FIELD

The disclosure relates generally to systems, devices, and methods for delivering one or more treatment agents. More specifically, aspects of the disclosure pertain to systems, devices, and/or methods for delivering one or more treatment agents, for example, prophylactic adhesives for hemostasis, via a medical device, such as an endoscope.

### BACKGROUND

For endoscopic procedures, treatment agents, such as prophylactic adhesives, may be used to promote natural healing by creating a protective layer that minimizes delayed bleeds, potential perforation, and stricture formation. These adhesives may be, for example, in the form of a gel, powder, patch, or film. Endoscopically delivering a patch, gel, patch, or film adhesive correctly may be difficult as target areas, especially in the esophagus, stomach, and colon, are often not smooth. Poor or improper delivery of the adhesive may impair visibility for the physician, for example, if the adhesive covers a portion of an endoscope lens. In addition, if the adhesive is not applied evenly across a surface, the uneven layer may not fully protect the surface or otherwise not promote healing. Furthermore, multiple deliveries of the adhesive may be required that may increase the duration and cost of the procedure.

These concerns may increase the duration, costs, and risks of the medical procedure. The systems, devices, and methods of this disclosure may rectify some of the deficiencies described above or address other aspects of the art.

### SUMMARY

This disclosure includes medical systems and devices for delivering one or more treatment agents (e.g., a biocompatible prophylactic adhesive) and methods of use thereof, e.g., methods of delivering one or more treatment agents to a target site of a patient, for example, to help heal an ulcer and/or to perform hemostasis.

In an example, a medical device may comprise: a sleeve; a conveyor movable relative to the sleeve; a treatment agent disposed within a cavity defined by the conveyor; and an actuator coupled to a first portion of the conveyor. The actuator may be movable relative to the sleeve. A second portion of the conveyor may be movable from a first position radially within the sleeve to a second position radially exterior to the sleeve, and at least a portion of the treatment agent may be movable from a first position within the cavity to a second position outside of the cavity.

Any of the aspects disclosed herein may include any of the following features, alone or in combination. The sleeve may include a proximal end and a distal end, and the conveyor may be configured to invert from the first position inside the sleeve to the second position outside the sleeve by inverting over the distal end of the sleeve. The distal end of the sleeve may be funnel-shaped. The funnel-shaped distal end of the sleeve may be biased closed by the actuator when the distal end of the sleeve is disposed radially within the actuator. The actuator may be a catheter. The conveyor may be composed of a first material, and the sleeve is composed of a second material, wherein the first material is more flexible than the second material. The conveyor may comprise a plurality of ring-shaped segments. The ring-shaped segments may fold over a distal end of the sleeve incrementally. Each of the ring-shaped segments may be formed of a plurality of prongs. The plurality of prongs of one ring-shaped segment may be connected to the plurality of prongs of a next ring-shaped segment. The sleeve may be formed of Nitinol. The conveyor may be a mesh. The treatment agent may be an adhesive gel. The medical device may be configured to be delivered to a treatment site through a working channel of an endoscope. A guidewire may guide the treatment agent to a target site.

In another aspect, a method of delivering a treatment agent may comprise: advancing a distal end of a medical device towards a target, the medical device comprising: a sleeve; a conveyor movable relative to the sleeve; a treatment agent disposed within a cavity defined by the conveyor; and an actuator coupled to a first portion of the conveyor. The actuator may be movable relative to the sleeve. The method of delivering the treatment agent may further comprise: actuating the actuator to move in a proximal direction. Actuating the actuator to move in the proximal direction may cause the cavity to move in a distal direction opposite the proximal direction, delivering the treatment agent within the cavity to a target site.

Any of the aspects disclosed herein may include any of the following features, alone or in combination. The method may further comprise removing and reloading the treatment agent into the cavity. The method may further comprise using a guidewire to guide the treatment agent to the target site.

In another aspect, a medical device may comprise: a handle that includes a movable portion; and an insertion portion. A distal portion of the insertion portion may include: a sleeve; a conveyor movable relative to the sleeve; a treatment agent disposed within a cavity defined by the conveyor; and an actuator coupled to the movable portion and to a first portion of the conveyor. The actuator may be movable relative to the sleeve such that: a second portion of the conveyor is movable from a first position radially inside the sleeve to a second position radially outside the sleeve, and the treatment agent may be movable from a first position within the cavity to a second position outside of the cavity.

Any of the aspects disclosed herein may include any of the following features, alone or in combination. The sleeve may include a proximal end and a funnel-shaped distal end. The conveyor may be configured to invert from the first position inside the sleeve to the second position outside the sleeve by inverting over the funnel-shaped distal end of the sleeve.

Any of the examples described herein may have any of these features in any combination. It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIG. 1A depicts a perspective view of a proximal portion of an exemplary medical system, including an exemplary medical device and an insertion device, according to one or more embodiments.

FIG. 1B depicts a perspective view of a distal portion of the exemplary medical system, according to one or more embodiments.

FIG. 2 depicts a cross-sectional view of a treatment agent delivery device, according to one or more embodiments.

FIG. 3A depicts a close-up view of a distal end of a treatment agent delivery device, according to one or more embodiments.

FIG. 3B depicts the treatment agent delivery device in operation, according to one or more embodiments.

FIGS. 4A-4C depict diagrammatic views of a treatment agent delivery device in operation, according to one or more embodiments.

FIGS. 5A-5C depict diagrammatic views of a treatment agent delivery device in operation, according to one or more embodiments.

### DETAILED DESCRIPTION

To endoscopically deliver a treatment agent(s) to difficult target areas, such as in the esophagus, stomach, and colon, the following systems, devices, and methods are disclosed. A sleeve may be used in conjunction with a conveyor and a wire to endoscopically introduce one or more treatment agents into a subject patient.

Reference is now made in detail to examples of this disclosure, aspects of which are illustrated in the accompanying drawings. The terms “proximal” and “distal” are used herein to refer to the relative positions of the components of an exemplary medical system and exemplary medical devices. When used herein, “proximal” refers to a position relatively closer to the exterior of the body of a subject or closer to a medical professional using the medical system or medical device. In contrast, “distal” refers to a position relatively further away from the medical professional using the medical system or medical device, or closer to the interior of the body of the subject. Proximal and distal directions are labeled with arrows marked “P” and “D”, respectively, throughout various figures. As used herein, the terms “comprises,” “comprising,” “has,” “having,” “includes,” “including,” or other variations thereof, are intended to cover a non-exclusive inclusion, such that a system, device, or method that comprises a list of elements does not include only those elements, but may include other elements not expressly listed or inherent thereto. Unless stated otherwise, the term “exemplary” is used in the sense of “example” rather than “ideal.” As used herein, the terms “about,” “substantially,” and “approximately,” indicate a range of values within +/−10% of a stated value. Wherever possible, the same reference numbers will be used throughout the drawings to refer to the same or like parts.

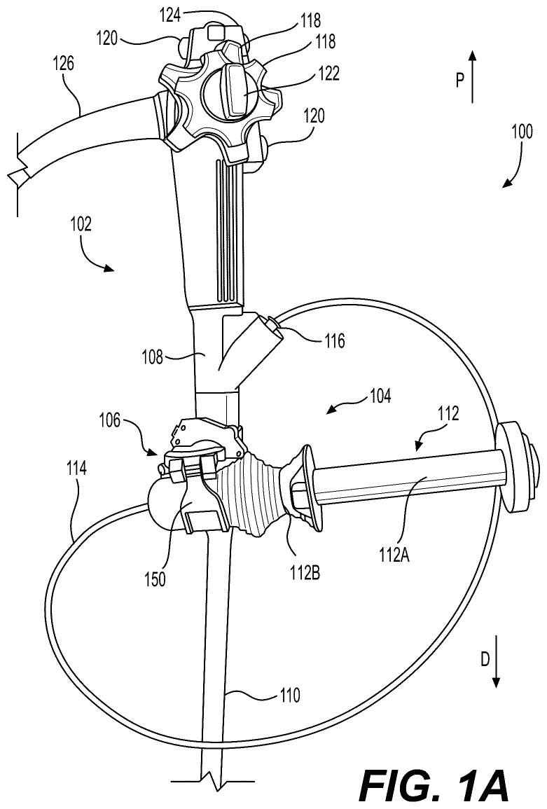

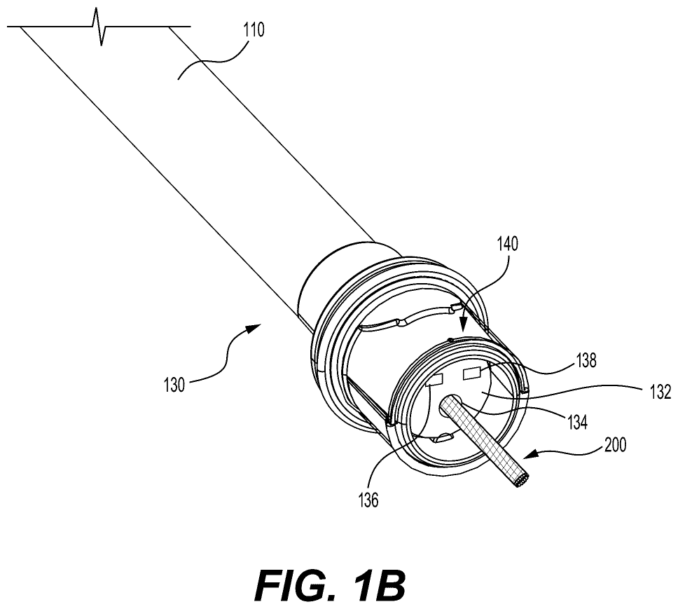

FIG. 1A illustrates an exemplary medical system 100, including a first medical device 102 and a second medical device 104. First medical device 102 may be an insertion device, and includes a handle 108 and an insertion portion 110. Second medical device 104 includes a handle 112 and an insertion portion 114. Handle 112 may be coupled to handle 108, for example, via one or more connectors 106, which may include one or more straps 150. Insertion portion 110 of first medical device 102 may be inserted into a subject, and insertion portion 114 of second medical device 104 may be delivered through a portion of insertion portion 110, for example, via a port 116 or other opening in handle 108 of first medical device 102.

As discussed in detail below, various aspects of second medical device 104 may be manipulated to endoscopically deliver one or more treatment agents, such as, for example, a patch, a gel, a film, an adhesive (e.g., a film adhesive), etc. Second medical device 104 may help to deliver the treatment agent(s) properly, for example, on a target area (e.g., in the esophagus, stomach, and colon) that is not smooth, which may help to promote healing on the target site. Furthermore, second medical device 104 may help to deliver the treatment agent(s) without impairing the visibility for the physician, for example, via an imaging device 138 on a distal end of insertion portion 110 of first medical device 102.

First medical device 102 includes handle 108 and insertion portion 110, which includes a distal portion 130 (FIG. 1B). One or more lumens may extend from handle 108 to the distal end of distal portion 130, for example, through insertion portion 110. As mentioned and as shown in FIG. 1A, handle 108 includes port 116 or another opening, which may connect through handle 108 to one or more lumens in insertion portion 110, such as a lumen 134 (see FIG. 1B). Moreover, handle 108 may include one or more knobs 118 and one or more actuators 120. For example, knobs 118 may be movable (rotatable or pivotable) to control the movement (e.g., deflection) of distal portion 130 of insertion portion 110. Handle 108 may also include one or more locking devices 122, for example, to secure the position of knobs 118, and thus secure the position of the distal portion of insertion portion 110. Additionally, actuators 120 may include one or more buttons or other types of actuators. Actuators 120 may be controlled to actuate the delivery of fluid, to actuate the application of suction, to actuate one or more illumination or imaging devices, etc. Furthermore, handle 108 may include one or more elevator actuators 124, for example, to control the position of an elevator positioned within the lumen of insertion portion 110.

First medical device 102 may include or be coupled to an umbilicus 126, for example, coupled to a proximal portion of handle 108. Umbilicus 126 may connect first medical device 102 to one or more of a control unit, a fluid source, a suction source, and/or other exterior devices, such as a monitor for viewing images from a camera.

Second medical device 104 includes handle 112 and insertion portion 114, and insertion portion 114 may be inserted through port 116. Additionally, handle 112 includes one or more movable portions 112A, for example, that is movable (e.g., translatable) relative to a stationary portion 112B to control one or more aspects of insertion portion 114. As discussed below, manipulation of movable portion 112A may control the delivery of one or more treatment agents. For example, movable portion 112A may be proximally retracted relative to stationary portion 112B to help deliver one or more treatment agents. Additionally, it is noted that FIG. 1B illustrates movable portion 112A in a proximally retracted configuration. Furthermore, various aspects of this disclosure may include different handles, for example, different from handle 112, in order to control the delivery of one or more treatment agents. Although not shown, handle 112 may include one or more additional controls, for example, similar to the control elements of handle 108.

In some aspects, first medical device 102 may be a gastroscope. Alternatively, first medical device 102 may be one of other similar insertion devices with a handle, such as, for example, endoscopes, ureteroscopes, duodenoscopes, endoscopic ultrasonography (“EUS”) scopes, colonoscopes, bronchoscopes, laparoscopes, arthroscopes, cystoscopes, aspiration scopes, etc. In some aspects, second medical device 104 may include a treatment agent delivery device 200 (FIG. 1B), for example, with a treatment agent (e.g., a prophylactic adhesive) that may be releasably deployed at a distal end of insertion portion 114. In these aspects, manipulation of handle 112 controls the deployment, delivery, or positioning of prophylactic adhesive, as discussed in further detail below.

FIG. 1B illustrates various features of distal portion 130 (i.e., a distal end) of insertion portion 110. Distal portion 130 may include, for example, a distal end face 132, and may include one or more of: a distal end or opening of lumen 134, one or more illumination device(s) 136 (e.g., one or more LEDs, optical fibers, etc.), and one or more imaging device(s) 138 (e.g., one or more cameras). Although not shown, distal portion 130 may also include one or more fluid delivery channels, one or more suction or negative pressure channels, etc. Lumen 134 may be one of one or more lumens defined in insertion portion 114 and may be used to introduce treatment agent delivery device 200, which in turn may be sized to fit into and/or be movable within lumen 134. In some aspects, medical system 100 may include an end cap 140, for example, coupled to distal portion 130 of insertion portion 110. Although not shown in detail, end cap 140 may include one or more treatment devices (e.g., one or more hemostatic patches).

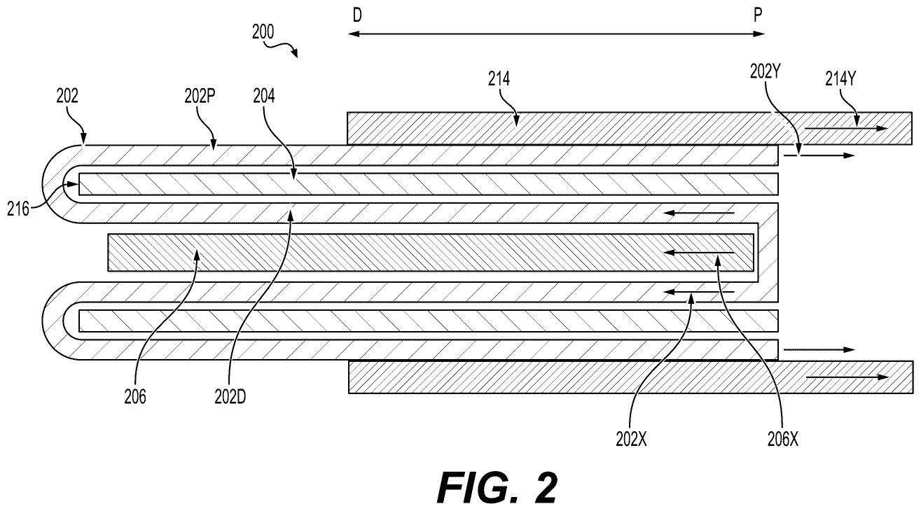

As depicted in FIG. 2, treatment agent delivery device 200 includes, among other elements, a conveyor 202, an inversion support sleeve 204, and an actuator 214. As discussed below, movement of the actuator 214 (e.g., via manipulation of one or more portions of handle 112) may control the movement of the conveyor 202, for example, relative to the sleeve 204, to pull the conveyor 202 in the delivery of the one or more treatment agents. Conveyor 202 may be formed of a flexible, stretchable material that may be inverted around a distal end of sleeve 204 when in operation for delivering a treatment agent 206, such as a prophylactic adhesive. Conveyor 202 includes portions that, in a first position, are disposed radially inside of sleeve 204, labeled 202D. In some aspects, conveyor 202 has a tube-like structure, and the diameter of the conveyor 202 while inside the sleeve 204 is smaller than the diameter of the sleeve 204. Due to the flexible, stretchable material, however, as conveyor 202 is inverted around or otherwise traverses a distal end 216 of sleeve 204, a distal portion of conveyor 202 is pulled in a proximal direction by actuator 214, labeled 202P. As will be described in more detail below, a portion 202D of conveyor 202 is disposed radially within sleeve 204 and a portion 202P of conveyor 202 is disposed radially outside of sleeve 204 in an initial configuration. The actuator 214 is connected to the portion 202P of conveyor 202 that is disposed radially outside of sleeve 204 in the initial configuration, and actuator 214 may be controlled by actuators disposed on handle 112. As actuator 214 is moved in a proximal direction as indicated by arrow 214Y, actuator 214 pulls the outer portion 202P of conveyor 202 in a proximal direction as indicated by arrow 202Y. The interior portion 202D of conveyor 202 is pulled in a distal direction, as indicated by arrow 202X. The movement of interior portion 202D in the distal direction pushes treatment agent 206 in the distal direction as well, as indicated by arrow 206X.

Sleeve 204 may be composed of a strong, flexible material, such as Nitinol, and is stationary relative to the conveyor 202 and the actuator 214. Sleeve 204 help to provide a support for the inversion of conveyor 202 at a distal end 216 as the actuator 214 pulls the outer portion of conveyor 202 in the proximal direction. It is noted that although FIG. 2 illustrates a distal portion of conveyor 202 spaced away from distal end 216, this is only for clarity in the Figure. In operation, the distal portion of conveyor 202 may abut at least a portion of distal end 216 as conveyor 202 is manipulated relative to sleeve 204 and distal end 216.

Actuator 214 may be a pull wire or a catheter coupled to the conveyor 202. The actuator 214 may be adhered to or otherwise coupled to the conveyor 202, and may be coupled at a proximal end of the conveyor 202 or any portion of the conveyor 202. Actuator 214 may be controlled via actuators on handle 112. The movement of actuator 214 in turn moves the conveyor 202, and movement of the conveyor 202 urges the treatment agent 206 distally. The movement of actuator 214 may incrementally move conveyor 202, and the incremental movement of conveyor 202 causes an incremental delivery of treatment agent 206.

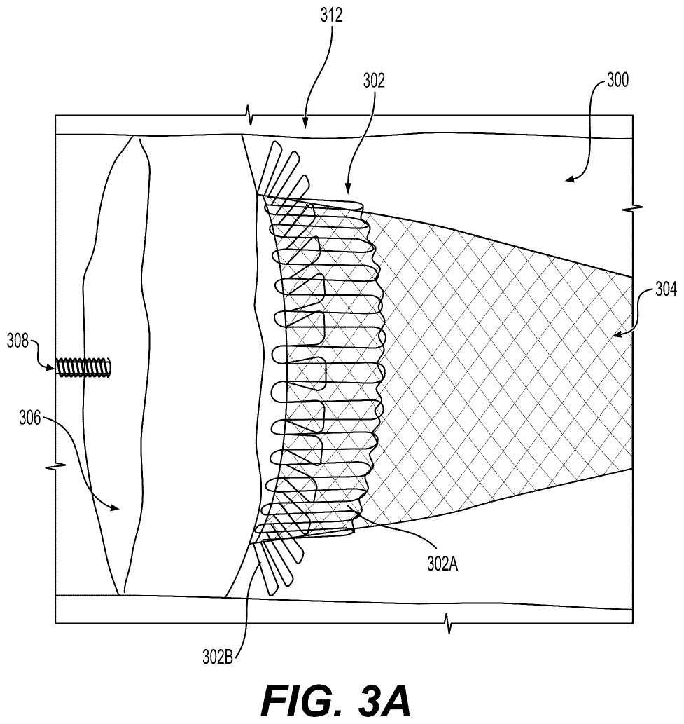

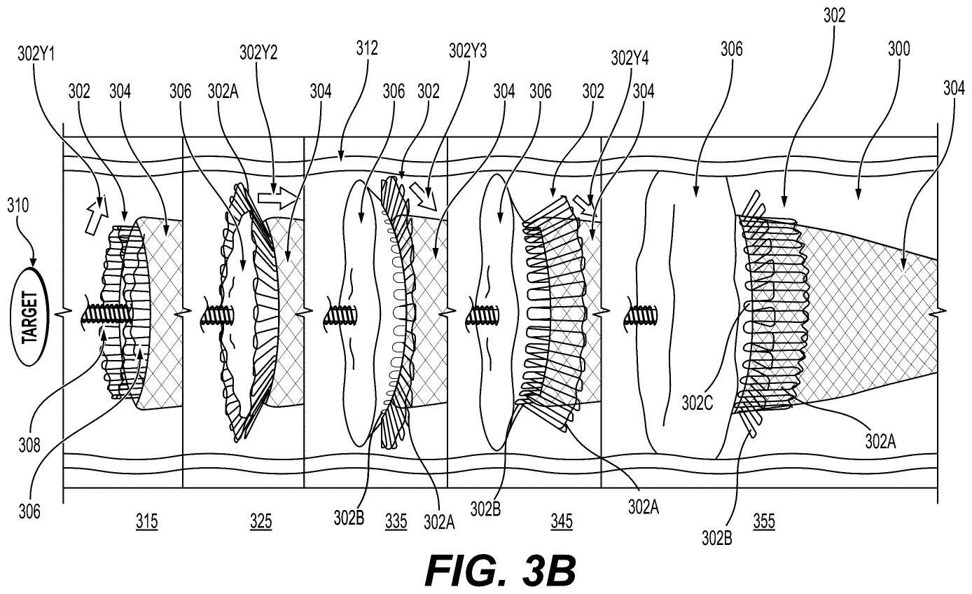

As mentioned above, a portion of conveyor 202 may be positioned and longitudinally slideable within sleeve 204, and arranged so that the distal end region of the flexible conveyor 202 doubles back over distal end 216 of the sleeve 204. Conveyor 202 is generally elongate and hollow and configured to slide and invert over distal end 216 of sleeve 204 when the distal end of conveyor 202 is pulled toward a proximal end of the sleeve 204, as will be described in more detail in FIGS. 3-5. The conveyor 202 may be tubular and may be formed of strips of material in segments as described in more detail below.

FIG. 3A illustrates a distal end of a treatment agent delivery device 300 inserted into a vessel lumen 312 of a patient, according to some embodiments. Delivery device 300 is substantially similar to delivery device 200, except as discussed below. The device 300 includes the inversion support sleeve 304 and the conveyor 302 that extends over the outer surface of the inversion support sleeve 304. The conveyor 302 may be referred to as a flexible tube or flexible conveyor and, as discussed above, may be attached at one end region to an actuator (e.g., actuator 214 shown in FIG. 2), which may be pull wire or pull tube, e.g., at the distal end region of the actuator. Pulling the actuator proximally inverts the flexible tube over the distal end of the inversion support sleeve 304 to push and incrementally deliver a treatment agent 306 (such as a prophylactic adhesive) in the vessel lumen 312, as shown in FIGS. 3A-3B. In operation, the amount of the material that may be delivered corresponds to the length of the conveyor 302. The movement of the actuator moves conveyor 302 in the distal direction, and movement of conveyor 302 urges the treatment agent 306 distally. The movement of the actuator may incrementally move conveyor 302, and the incremental movement of conveyor 302 causes an incremental delivery of treatment agent 306.

In use, a guidewire 308 may be configured to slide through or adjacent to the conveyor 302. In some aspects, guidewire 308 may form part of the apparatus and/or may otherwise help to allow positioning and/or support of delivery device 300 (e.g., in the delivery of an insertion device). Guidewire 308 may not impair or otherwise interfere with the operation of conveyor 302 delivering an object, such as a prophylactic adhesive out of the body of the conveyor 302. Guidewire 308 may be disposed within conveyor 302 and attached to treatment agent 306 to help provide cohesion with the treatment agent and guide the treatment agent toward a target 310. Target 310 may be, for example, an ulcer in the esophagus, stomach, or colon. In some embodiments, the delivery of the treatment agent may be accomplished without the use of a guidewire.

FIG. 3B demonstrates an example of the treatment agent delivery device 300 in use. FIG. 3B includes five stages of the device in use, from the arrival of the device 300 at a position adjacent to a target site 310 to the delivery of the treatment agent 306 to the target site 310. Before the treatment agent delivery device 300 is advanced to the target site (e.g., through the working channel or insertion portion 110 of first medical device 102), a treatment agent 306 may be inserted into the device 300, positioned within conveyor 302. Conveyor 302 may be substantially retracted into the sleeve 304 by pulling a guidewire 308 or, in some instances, by pushing on an actuator, such as an actuator substantially similar to actuator 214 shown in FIG. 2. When prepared for delivery, the treatment agent delivery device 300 is inserted into first medical device 102 (e.g., via port 116) and through insertion portion 110. Then, insertion portion 110 may be inserted into, for example, a target patient.

At a first stage 315, the prophylactic delivery device 300 has been navigated endoscopically to target 310. As discussed above, first medical device 102 may be a gastroscope with an insertion portion 110 (see FIG. 1A, 1B), and second medical device 104 may be a treatment agent delivery device 300 with a treatment agent releasably deployed at a distal end of insertion portion 110. Manipulation of handle 108 may control the positioning of the insertion portion 110 of the first medical device, and manipulation of handle 112 may control the deployment, delivery, or positioning of one or more treatment agents.

While the treatment agent delivery device 300 is being positioned, treatment agent 306 is substantially entirely within the conveyor 302, and the conveyor 302 is substantially entirely within the sleeve 304. In some embodiments, the conveyor 302 may have a portion substantially within the sleeve 304 and a portion, such as end segment 302A protruding out of the sleeve 304. An actuator may be arranged around conveyor 302 (such as actuator 214, shown in FIG. 2) such that a user may pull on the actuator in a proximal direction (to the right in FIG. 3B), which in turn causes a pulling of the distal end of conveyor 302 to be inverted from a distal direction to a proximal direction around a distal end of sleeve 304, in a radially outward motion as shown by arrow 302Y1. The actuator may be coupled to end segment 302A by a mechanical link, an adhesive, or by other coupling mechanisms. In some embodiments, a plurality of segments 302A, 302B, etc., may protrude from the distal end of the sleeve 304 and the actuator 214 may be coupled to more than one of the segments 302A, 302B, etc., of the conveyor 302. In yet other embodiments, the actuator 214 may be coupled to one or more segments 302B, etc., that does not include the end segment 302A. Guidewire 308 may be directed to the target 310 to aid in delivering the treatment agent accurately to the target 310. Additionally, the positioning or orientation of delivery device 300 may also be controlled by positioning, deflecting, or otherwise controlling the position of the distal end of the insertion device (e.g., distal portion 130, FIG. 1B), for example, via manipulation of one or more of the actuators on the handle of the insertion device.

Next, at a stage 325, a first conveyor segment 302A has been pulled out of the sleeve 304 and has begun to fold toward the proximal end of the sleeve 304, as shown by arrow 302Y2. Each segment 302A, 302B, etc., of the conveyor 302 may be formed of a series of prongs that are arranged in a ring and link with the next segment, the series of prongs formed into a ring-shaped segment. The connected segments 302A, 302B, etc. form a flexible hollow tube configured to be inverted around the sleeve 304 as they protrude from the distal end of the sleeve 304 and are pulled in the proximal direction by an actuator 214. As the conveyor 302 is folded over the distal end of the sleeve 304 and pulled in the proximal direction, the treatment agent 306 disposed within the conveyor 302 is pushed out by the back end of the conveyor 302 and the friction with the surface of the conveyor and emerges from the delivery device 300. Guidewire 308 may help to direct the treatment agent 306 to the target 310 as treatment agent 306 is incrementally delivered from the delivery device 300.

Furthermore, at a stage 335, a first segment 302A is in the process of folding toward the proximal end, while a second segment 302B has begun to emerge from inside the sleeve 304. As the segments continue to move within the sleeve in a distal direction, more of treatment agent 306 emerges from the conveyor 302 and is directed toward the target 310. During these stages, sleeve 304 and guidewire 308 are substantially stationary. In some aspects, sleeve 304 and/or guidewire may be adjusted, for example, to help maintain proper positioning of the delivery device 300.

At a stage 345, first segment 302A has substantially folded over, as demonstrated by the solid arrows, such that its previously distal end is now facing in the opposite direction and is being pulled toward the proximal end as a result of the continued pulling effect of actuator 214, which may be or include a catheter. Each segment 302A, 302B, etc. is formed of strong but flexible material. As such, each segment 302 may expand to a larger size to wrap around sleeve 304 or contract to fit into sleeve 304.

At stage 355, more of treatment agent 306 is delivered from the conveyor 302. Furthermore, segment 302B has substantially followed the path of segment 302A, with a third segment 302C beginning to emerge from within sleeve 304. This process may be continued until the treatment agent is completely delivered from within the conveyor 302 and applied to the target 310. In other instances, some of the treatment agent 306 may be delivered to a first target 310, and the delivery device 300 may be repositioned to deliver a remaining amount of treatment agent 306 to other targets. After the treatment agent has been successfully delivered to target 310, the guidewire 308 may be retracted by an operation of a control or actuator on handle 108 or handle 112.

After successful delivery of a treatment agent 306, the delivery device 300 may be retracted through lumen 134 and insertion port 116, removing the delivery device 300 from a patient. A user may then prepare a second treatment agent 306 in the delivery device 300 and, without having removed the first medical device from the patient, direct the treatment agent delivery device 300 to a second target 310 and repeat the stages 315 through 355 to deliver a second treatment agent 306 (either the same or different type of treatment agent as the first treatment agent) to the patient. This process may be repeated as many times as necessary before withdrawing the first medical device from the patient, allowing for the treatment of multiple ulcers or other targets requiring hemostasis without removing the first medical device, e.g., the gastroscope, making the process more efficient and safer for the patient by reducing the number of times a scope needs to be removed and reinserted. The delivery of treatment agent 306 may be observed and monitored using the one or more imaging devices 138 on the distal end of the insertion portion of the first medical device 104.

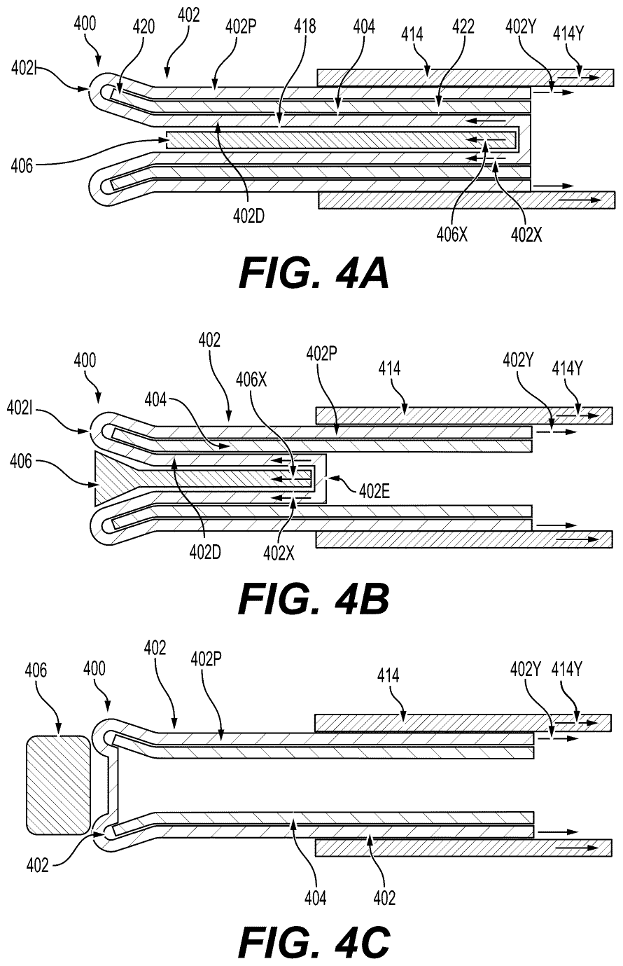

FIGS. 4A-4C depict various diagrammatic cross-sectional views of a delivery device 400 in operation, according to one or more embodiments, to help better illustrate the mechanism for delivery. Delivery device 400 is substantially similar to delivery device 200, except as discussed below. FIG. 4A may represent the delivery device 400 in an initial state, with a treatment agent 406 disposed within a cavity 418 defined by conveyor 402, specifically an interior portion 402D of conveyor 402. Interior portion 402D is disposed radially inside of sleeve 404, and is configured such that, as an actuator 414 is pulled in a proximal direction 414Y (to the right in FIGS. 4A-4C), an exterior portion 402P of conveyor 402 is pulled in the proximal direction 402Y, while the interior portion 402D is pulled in the distal direction (to the left in FIGS. 4A-4C), the interior portion being pulled in the proximal direction until it reaches a distal end 416 of inversion sleeve 404. When the actuation of the actuator 414 has urged a given portion of interior portion 402D distally to the distal end 416 of the inversion sleeve, the portion of the conveyor 402 inverts upon the distal end 416, effectively forming an inversion portion 4021 of the conveyor 402.

In some embodiments, such as that shown in FIGS. 4A-4C, the distal end of sleeve 404 may include an angled, flared, or funneled portion 420 that flares radially outward from the remainder of the sleeve 404, such that the distal end of sleeve 404 is funnel-shaped. In some aspects, the remainder of sleeve 404 may be a generally cylindrical portion 422. The funneled portion 420 may help support the inversion of the conveyor 402 at the inversion portion 4021, for example, as treatment agent 406 is deployed when actuator 414 is pulled proximally. Furthermore, the funneled portion 420 provides a large surface area to apply the treatment agent onto a target site. As such, for larger lesions (such as those, e.g., greater than 3 cm in diameter), the treatment agent may be applied quickly, which may help to procedure time. The funneled portion 420 may be at least partially resilient, for example, such that funneled portion 420 is biased closed by the actuator 414 until actuator is retracted proximally such that funneled portion 410 is revealed or otherwise exposed. Thus, when inserting the treatment agent delivery device 400 into a patient's lumen 412 via the lumen 134 in the insertion portion 110, the funneled portion 420 is biased to a position flush with the cylindrical portion 420. Once the delivery device 400 arrives at a target, a user may actuate actuator 414 via controls on handle 112 to begin to pull back the actuator 414, revealing the funneled portion 420 such that funneled portion 420 opens or otherwise expands into the funnel position shown in FIG. 4A.

As shown in FIG. 4B, as the conveyor 402 continues to move in response to an actuation, an end portion 402E of the conveyor 402 translates within the interior of sleeve 404 in a distal direction. As end portion 402E translates in the distal direction, conveyor 402 pushes the treatment agent 406 stored in the cavity 418 defined by the conveyor 402 toward the distal end 416. As the conveyor 402 approaches its inversion portion 4021, the portion of the conveyor 402 inverts and begins moving in a proximal direction, while the treatment agent 406 begins to be discharged from the delivery device 400.

FIG. 4C depicts the delivery device 400 with a treatment agent 406 completely delivered after the actuator 414 has been actuated to a point at which the end portion 402E of conveyor 402 has arrived at the distal end 416 of the sleeve. The end portion 402E may be constructed of a different material than the remainder of the conveyor 402, or otherwise be constructed such that it does not invert over the distal end 416. When the end portion 402E arrives at distal end 416, actuator 414 may no longer be actuated, and delivery device 400 (e.g., via the medical device 104) may be removed from the patient.

In these aspects, and as described with reference to FIG. 2 above, as an actuator 414 is moved in a proximal direction 414Y, the actuator 414 pulls the outer portion 402P of conveyor 402 in a proximal direction as indicated by arrow 402Y. The interior portion 402D of conveyor 402 is pulled in a distal direction, as indicated by arrow 402X. The movement of interior portion 402D in the distal direction pushes treatment agent 406 in the distal direction as well, as indicated by arrow 406X.

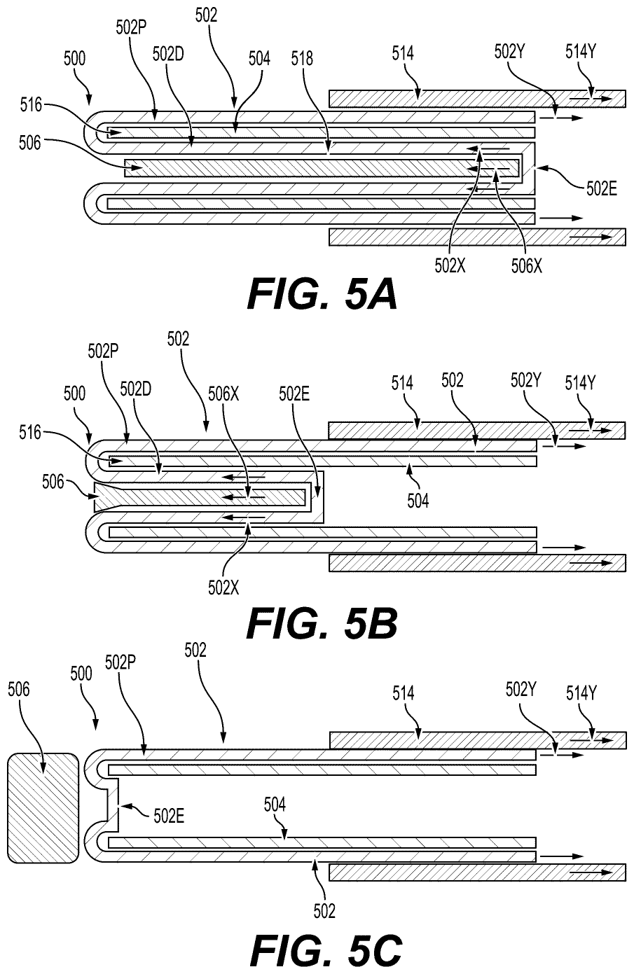

FIGS. 5A-5C also depict various diagrammatic cross-sectional views of a treatment agent delivery device 500 in operation, according to one or more embodiments. The primary difference between the device 500 described in FIGS. 5A-5C and the device 400 described in FIGS. 4A-4C is the distal end 516 of the sleeve 504 in the embodiments shown in FIGS. 5A-5C does not include the funneled portion 420 of the device 400 described in FIGS. 4A-4C.

Similarly to FIG. 4A above, FIG. 5A may represent the delivery device 500 in an initial state, with a treatment agent 506 disposed within a cavity 518 defined by conveyor 502, specifically an interior portion 502D of conveyor 502. Conveyor 502 may further be formed of a mesh, which allows for tuning of the mesh material properties such as flexibility and durability. This allows for treatment agents with differences in elasticity and cohesion to be delivered with the same delivery device with specified mesh materials. Meshes may vary in size and shape, and material properties of the mesh. Different types of meshes may be appropriate for different types of treatment agents. Interior portion 502D is disposed radially inside of sleeve 504, and is configured such that, as actuator 514 is pulled in a proximal direction 514Y (to the right in FIGS. 4A-4C), an exterior portion 502P of conveyor 502 is pulled in the proximal direction 502Y, while the interior portion 502D is pulled in the distal direction 502X (to the left in FIGS. 4A-4C). The interior portion 502D may be pulled in the distal direction until it reaches a distal end 516 of inversion sleeve 504. When the actuation of the actuator 514 has pulled a given portion of interior portion 502D distally to the distal end 516 of the inversion sleeve, the portion of the conveyor 502 inverts upon the distal end 516, effectively forming an inversion portion 5021 of the conveyor 502.

As shown in FIG. 5B, as the actuator 514 is continued to be pulled back in the proximal direction, an end portion 502E of the conveyor 502 translates within the interior of sleeve 504 in a distal direction. As end portion 502E translates in the distal direction, it pushes the treatment agent 506 stored in the cavity 518 defined by the conveyor 502 toward the distal end 516. As the conveyor 502 approaches its inversion portion 5021, the portion of the conveyor 502 inverts and begins moving in a proximal direction, while the treatment agent 506 begins to be discharged from the delivery device 500.

FIG. 5C depicts the delivery device 500 with a treatment agent 506 completely delivered after the actuator 514 has been actuated to a point at which the end portion 502E of conveyor 502 has arrived at the distal end 516 of the sleeve. The end portion 502E may be constructed of a different material than the remainder of the conveyor, or otherwise be constructed such that it does not invert over the distal end 516. When the end portion 502E arrives at distal end 516, actuator 514 may no longer be actuated.

As described with reference to FIG. 2 above, as an actuator 514 is moved in a proximal direction 514Y, the actuator 514 pulls the outer portion 502P of conveyor 502 in a proximal direction as indicated by arrow 502Y. The interior portion 502D of conveyor 502 is pulled in a distal direction, as indicated by arrow 502X. The movement of interior portion 502D in the distal direction pushes treatment agent 506 in the distal direction as well, as indicated by arrow 506X.

The treatment agent delivery device 500 may then be withdrawn, for example, proximally from the insertion portion 110 of the first medical device 102 via the lumen 134. Once removed, the delivery device 500 may be replaced or reloaded with a new treatment agent 506. Another or the same device 500 may be re-inserted into an insertion device and/or into the patient (e.g., with device 500 in the initial state shown in FIG. 5A). This process may be repeated as many times as necessary for a patient with the advantage that the first medical device 102 need not be removed in between deliveries of treatment agents.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device comprising:
a sleeve;
a conveyor movable relative to the sleeve;
a treatment agent disposed within a cavity defined by the conveyor; and
an actuator coupled to a first portion of the conveyor, wherein the actuator is movable relative to the sleeve; wherein:
a second portion of the conveyor is movable from a first position radially within the sleeve to a second position radially exterior to the sleeve, and
at least a portion of the treatment agent is movable from a first position within the cavity to a second position outside of the cavity.

2. The medical device of claim 1, wherein the sleeve includes a proximal end and a distal end, and wherein the conveyor is configured to invert from the first position inside the sleeve to the second position outside the sleeve by inverting over the distal end of the sleeve.

3. The medical device of claim 2, wherein the distal end of the sleeve is funnel-shaped.

4. The medical device of claim 3, wherein the funnel-shaped distal end of the sleeve is biased closed by the actuator when the distal end of the sleeve is disposed radially within the actuator.

5. The medical device of claim 1, wherein the actuator is a catheter.

6. The medical device of claim 1, wherein the conveyor is composed of a first material, and the sleeve is composed of a second material, wherein the first material is more flexible than the second material.

7. The medical device of claim 1, wherein the conveyor comprises a plurality of ring-shaped segments.

8. The medical device of claim 7, wherein the ring-shaped segments fold over a distal end of the sleeve incrementally.

9. The medical device of claim 8, wherein each of the ring-shaped segments is formed of a plurality of prongs.

10. The medical device of claim 9, wherein the plurality of prongs of one ring-shaped segment are connected to the plurality of prongs of a next ring-shaped segment.

11. The medical device of claim 1, wherein the sleeve is formed of Nitinol.

12. The medical device of claim 1, wherein the conveyor is a mesh.

13. The medical device of claim 1, wherein the treatment agent is an adhesive gel.

14. The medical device of claim 1, wherein the medical device is configured to be delivered to a treatment site through a working channel of an endoscope.

15. The medical device of claim 1, further comprising a guidewire to guide the treatment agent to a target site.

16. A method of delivering a treatment agent, the method comprising:
advancing a distal end of a medical device towards a target, the medical device comprising:
a sleeve;
a conveyor movable relative to the sleeve;
a treatment agent disposed within a cavity defined by the conveyor; and
an actuator coupled to a first portion of the conveyor, the actuator movable relative to the sleeve;
actuating the actuator to move in a proximal direction, wherein actuating the actuator to move in the proximal direction causes the cavity to move in a distal direction opposite the proximal direction, delivering the treatment agent within the cavity to a target site.

17. The method of claim 16, further comprising:
removing and reloading the treatment agent into the cavity.

18. The method of claim 16, further comprising:
using a guidewire to guide the treatment agent to the target site.

19. A medical device, comprising:
a handle, wherein the handle includes a movable portion; and
an insertion portion, wherein a distal portion of the insertion portion includes:
a sleeve;
a conveyor movable relative to the sleeve;
a treatment agent disposed within a cavity defined by the conveyor; and
an actuator coupled to the movable portion and to a first portion of the conveyor, wherein the actuator movable relative to the sleeve such that:
a second portion of the conveyor is movable from a first position radially inside the sleeve to a second position radially outside the sleeve, and
the treatment agent is movable from a first position within the cavity to a second position outside of the cavity.

20. The medical device of claim 19, wherein the sleeve includes a proximal end and a funnel-shaped distal end, wherein the conveyor is configured to invert from the first position inside the sleeve to the second position outside the sleeve by inverting over the funnel-shaped distal end of the sleeve.
